(19) United States
(12) Patent Application Publication
(10) Pub. No.: US 2019/0298757 A1
(43) Pub. Date: Oct. 3, 2019

(54) EDIBLE SEMI-SOLID COMPOSITION FOR USE IN PATIENTS UNDERGOING ENDOSCOPY INCLUDING COLONOSCOPY

(71) Applicants: UNIVERSITY OF COPENHAGEN, Copenhagen K (DK); SJÆLLANDS UNIVERSITETSHOSPITAL, Køge (DK)

(72) Inventors: Daniel BAR-SHALOM, Kokkedal (DK); Peter VILMANN, Klampenborg (DK); Anette MÜLLERTZ, Charlottenlund (DK); Jacob Kolstrup ZEDERKOF, København V (DK); Hanne HOLM, Havdrup (DK); Jette JACOBSEN, Ølstykke (DK)

(73) Assignees: UNIVERSITY OF COPENHAGEN, Copenhagen K (DK); SJÆLLANDS UNIVERSITETSHOSPITAL, Køge (DK)

(21) Appl. No.: 16/302,578

(22) PCT Filed: May 5, 2017
(86) PCT No.: PCT/EP2017/060748

§ 371 Date: Nov. 16, 2018

(30) Foreign Application Priority Data
May 18, 2016 (DK) PA 2016 70326

(51) Int. Cl. A61K 31/765; A61K 47/36; A61K 9/10; A61K 31/7016; A61K 31/723; A61K 31/732; A61K 31/737; A61K 36/04; A61K 45/06; A61P 1/10; A61K 9/00; A61K 47/46

(52) U.S. Cl. CPC A61K 31/765; A61K 47/36; A61K 9/10; A61K 31/7016; A61K 31/723; A61K 47/46; A61K 31/737; A61K 36/04; A61K 45/06; A61P 1/10; A61K 9/0056; A61K 31/732

(57) ABSTRACT

The present invention relates to a GI-cleansing composition comprising i) one or more gelling or swelling agents, ii) one or more laxative agents, iii) one of more taste-improving agents, and iv) water.

### FIELD OF THE INVENTION

The present invention relates to a semi-solid composition for use in cleansing the intestines in preparation to imaging and other procedures requiring an empty, visually clean environment. The composition is an improvement compared to conventional compositions in that the laxative and cleansing active ingredients are concentrated in a composition of a relative small volume and at the same time, the composition has a structure and consistency that resemble normal food such as a pudding or dessert optionally with a crème sauce.

In a further embodiment of the invention, the same composition, in smaller amounts can be used as a laxative for children and adults suffering from constipation. Moreover, it may also be used as a weight loss or anti-obesity composition both for cosmetic purposes or for medical purpose.

### BACKGROUND OF THE INVENTION

Colorectal (bowel) cancer is the second leading cause of cancer related deaths in the US, UK and Europe. Despite results of treatment showing a gradual improvement in the UK over the past 30 years, 5-year survival is still only around 50%.

A colonoscopy is a diagnostic procedure that is used to evaluate disorders of the large intestine as well as for colorectal cancer screening. Colonoscopies have been demonstrated to reduce the incidence of colorectal cancer (CRC, both ascending and descending sections) by detection and removal of precancerous lesions known as polyps (small, abnormal, unusually benign growth of tissue. However, the diagnostic accuracy of the colonoscopy hugely depends on the quality of the pre-procedure bowel preparation (the quality of bowel cleansing is usually assessed by the quantity of solid and liquid stool matter in the lumen and measured on a 5-point or a 10 point scale; Boston scale BBPS). Poor quality bowel cleansing has a significant negative impact on the detection of polyps (of any size) and polyps are more likely to be missed (nevertheless the detection of colon cancer itself was not reduced). Poor bowel preparation resulted in longer, more difficult procedures and a lower percentage of completed procedures. The quality of cleansing was only high in 74% of patients (Froehlich et al., 2005). Unfortunately up to 20-25% of all patients has inadequate bowel cleansing

However, the pre-procedure bowel cleansing is unpleasant and often found to be worse than the colonoscopy itself. The diagnostic accuracy of a colonoscopy is directly linked to the quality of bowel cleansing.

Patients undergoing colonoscopy (here understood to include other endoscopic procedures as well) are faced with long periods without food (typically up to 24 hours before colonoscopy) and the bowel needs to be clean as the cleansing quality critically determines quality, difficulty, speed and completeness of colonoscopy and to ensure that diseases or signs of diseases in the bowel are discovered by the procedure.

Several bowel cleansing compositions and regimens are available. In a recent review by Johnson et al. in The American Journal of Gastroenterology, 2014, 1-14 it is recommended in general that efficacy has a higher priority than tolerability as inadequate cleansing is not desired. Most of the colonoscopy compositions available are based on the use of polyethylene glycol 3350.

The gold standard is a split-dose regime of 4 litres of PEG-ELS (Polyethylene glycolelectrolyte lavage solution) and is the most recommended preparation method pre-colonoscopy as it gives high quality cleansing (Johnson et al., 2014). The preparation fluid is usually split with around half being taken the day before the colonoscopy and the other half the morning of the colonoscopy, and a clear liquid diet is usually necessary for one of more days prior to procedure—often leaving patients hungry and lacking in energy. One important point is that many patients report that bowel cleansing preparation was worse than the procedure itself (which is surprising when you consider the procedure) (McCray and Balaban, 2007; Ko et al., 2007). The perception of the patients influences the compliance and the outcome of the cleansing (Menes et al., 2014)

Other compositions based on phosphate, sodium sulfate or sodium picosulfate are also available, but recently some phosphate-based compositions were withdrawn from the US market due to the risk of renal damage. A common feature of most of the colonoscopy compositions available is that they must be administered in large volumes, normally in a volume of 2 or 4 liters split into a two-fold dosage regime, where one dose is given the day before colonoscopy and the second dose is given 4-6 hours before colonoscopy. The treatment is not pleasant for the patient; as McCray and Balaban say: “Traditionally, patients undergoing colonoscopy have faced long periods without food, followed by an oral preparation consisting of seemingly unending amounts of unpleasant tasting liquids, culminating in large volumes of liquid stool and a sore bottom”.

Patient compliance is another issue as it is difficult for the patients to intake so large volumes without having access to any food. Accordingly, some patients may not ingest the whole dose and thereby they may risk that the colonoscopy becomes ineffective due to non-optimal cleansing of the bowel. Moreover, the patient must be in fasted state, which is a challenge for most of the patients, as they must intake huge volumes of a liquid composition, which triggers the desire for normal food.

Accordingly, there is a need to develop novel bowel cleansing compositions. Ideally, the compositions containing the gastro-intestinal (GO-cleansing agent should be of much smaller volume, the taste should be improved and the structure or texture of the composition should be pleasant and easy to eat, notably in a structure resembling a dietary product i.e. like pudding or a pudding with a crème sauce.

### DETAILED DESCRIPTION OF THE INVENTION

The present invention provides such a GI-cleansing composition. In the present context, the term GI-cleansing composition indicates a composition that enables cleansing of at least that part of the gastrointestinal tract, which is undergoing inspection. Thus, in case of colonoscopy, at least the colon must be cleansed. A composition of the invention is highly concentrated with respect to laxative agents, which means that the patient undergoing colonoscopy shall not ingest large volumes of a bad-tasting composition. Moreover, a composition of the invention has a texture like pudding, which means that the patient undergoing colonoscopy is not be subjected to ingest large volumes of foul-tasting liquids, but will be able to intake a composition that texture-wise is comparable to a dessert like a pudding. This is believed to be an advantage as the patient normally must be in fasted condition for about 24 hours before the colonoscopy is performed. Another advantage is that the patient can ingest the composition together with plain water, sparkling water or other allowed beverages, i.e. beverages that do not in themselves taste badly or contain undigestable fiber such as unfiltered orange juice. It is important that the patient drinks 1-2 liter of water or the like while or after the GI-composition has been eaten. Therefore, it is an advantage that the GI-composition triggers a desire for intake of water. All in all, the volume of the composition of the active laxative agents is markedly reduced, the taste is markedly improved and the patient ingests a composition that has a texture like a dessert, which minimizes the negative effects of only being allowed to intake liquids.

The present invention provides a GI-cleansing composition comprising
i) one or more gelling or swelling agents,
ii) one or more laxative agents,
iii) one of more taste-improving agents, and
iv) water
   wherein the composition is in the form of a gel having pudding-like structure. The gel may optionally exert syneresis.

The content of said one or more laxative agents in a composition of the invention corresponds to from 20% w/w to 100% of the dose required for obtaining cleansing of the intestines, and the volume of the composition per dose is from about 50 mL to about 500 mL. It is preferred that the volume per dose is from about 50 mL to about 300 mL, notably from about 100 mL to about 250 mL. If, for example, the composition contains 20% of the total dose required, then the composition must be administered five times before endoscopy and if the composition contains 25% of the total dose required, then the composition must be administered four times before endoscopy etc. As described herein it is preferred that the total dose is split into 2-4 compositions, where 1-2 compositions are administered the day before endoscopy and the remaining 1-2 compositions are administered in the morning just before endoscopy. The split of the dose required also enables the possibility of using compositions with a different taste.

Suitable gelling or swelling agents are e.g. gellan gum, agar-agar, guar gum, konjak, alginic acid, sodium alginate, potassium alginate, ammonium alginate, calcium alginate, carrageenan, processed eucheuma seaweed, locust bean gum (carob), tragacant, acacia gum, xanthan gum, karaya gum, tara gum, pectin, chitosan, gelatin, psyllium husk, modified celluloses such as methylcellulose, sodium carboxymethylcellulose, cross-linked sodium carboxymethylcellulose, hydroxypropylcellulose, hydroxypropylmethylcellulose, ethylmethylcellulose, enzymatically hydrolysed carboxymethylcellulose, polyvinylpyrrolidone, starch, modified starches, or mixtures thereof.

As seen from the examples herein a very suitable gelling or swelling agent is carrageenan or carrageenan in combination with a gum such as locust bean gum, which is believe to strengthen the gel formed.

US 2010/255122 describes edible composition comprising gelatin. However, the patient still need to intake large volumes in order to obtain the desired response.

Some of the gelling or swelling agent may be an agent that is capable of exerting syneresis. Syneresis is a phenomenon that is observed when a gel stands for some time, shrinks and some of its liquids are pressed out. In a composition of the invention, this is believed to be an advantage in that the composition then appears as a pudding surrounded by or floating in a sauce, i.e. just like e.g. crème caramel. Syneresis is thought to be due to a continued coarsening of the matrix or fibrous structure of a gel with a consequent squeezing-out effect. Thus, syneresis is a phenomenon related to a gel structure, which makes gelling agents of primary interest in the present context. However, in the literature, there is often no clear distinguishing between swelling and gelling agents and both types of agents are contemplated to be suitable to use in a composition of the present invention.

Gelling agents that can exert syneresis include—but not limited to—gellan, guar gum, agar-agar, and gelatin by themselves or when in combination with other ingredients. It is also contemplated that the microparticles in the water-in-water emulsions (mentioned in the following and containing PEG) are syneretic.

Normally, the gelling or swelling agent(s) are present in a composition of the invention in a concentration that is sufficient to obtain a gel or gel-like structure and to obtain a texture like a pudding. The specific concentration will depend on the specific gelling or swelling agent used, but is normally in a range of from 1% to about 15% w/w specifically, when gellan is used, gellan is present in a composition of the invention in a concentration of from about 0.5 to 7.5% w/w. When guar gum is employed, normally the concentration is from about 1 to about 10% w/w. When agar is employed, the concentration is from about 1 to about 10% w/w. The individual gelling or swelling agent may be purchased in various qualities, which means that the concentration of the individual gelling or swelling agent may be adjusted to ensure that the desired viscosity is obtained. A mentioned herein before, the viscosity corresponds to the viscosity of a pudding, i.e. it is not a liquid neither is it a solid, but a semi-solid composition.

As seen from the examples, herein the gelling or swelling agent may also be one, which contribute to obtaining a composition, which is in the form of a water-in-water emulsion (w/w emulsion). A w/w emulsion is an emulsion having two aqueous phases, where one of the phase is the continuous phase and the other phase is the disperse phase. A w/w emulsion may be formed when two hydrophilic phases (two immiscible phases) are mixed. w/w emulsions are much less known that the typical oil-in-water emulsions (or vice versa). The kinetic stability of the w/w emulsion is difficult to control as amphipathic molecules (having both a hydrophilic and hydrophobic part), do not adsorb onto the w/w interface, hence surfactants cannot be used (Esquena, 2016). Surfactants usually act to prevent fusion when the droplets collide. However, water-in-water emulsions cannot be stabilised by surfactants so instead they may be stabilised by gelling one or both of the phases. It has also recently been found that the water-in-water emulsion can be stabilised by the addition of protein particles that created a monolayer of protein particles at the water-water interface of the emulsion (Nguyen et al., 2013).

As seen from the examples herein, a suitable composition is found when carrageenan is used as a swelling agent and PEG 3350 is used as a laxative agent. Such a composition may be in the form of a w/w emulsion, where the carrageenan-containing phase is the continuous phase and the PEG 3350-containing phase is the disperse phase. An advantage of having the composition in the form of an emulsion, where the “bad-tasting” PEG 3350 is in the disperse phase, may be that it contributes to the taste-masking of the bad taste of PEG 3350.

Carrageenan is suitable for vegetarians and avoids the issues of the animal-derived gelatin. Carrageenans are anionic linear polymers comprising of 1,3α-1,4β-galactans units. In an ionic solution κ-carrageenan (as used in the examples self-associates to form a rigid, helical structure and hence a thermoreversible water gel. Other carrageenans may also be used such as iota-carrageenan.

When eg carrageenan is used in a composition of the invention, it is normally used in a concentration of from about 0.1-10% w/w, preferably from about 0.1 to 5% w/w. As seen from the examples herein, carrageenan may be used together with another gelling or swelling agent, notably Carob (locust bean gum), to strengthen the strength of the gel. When used together, the concentration of carrageenan may be reduced to be in a range of from about 0.1-2% or from 0.1%-1% w/w. The concentration of carob is in the same range as the concentration of carrageenan. As seen from the examples herein, the composition becomes less brittle, more elastic and the gel becomes overall stronger when carob is used together with carrageenan compared with a composition containing carrageenan, but without carob. Thus, as demonstrated in the examples herein use a gel containing 0.2% w/w carrageenan and 0.2% w/w carob is just as effective regarding gel strength as a gel containing 0.6% w/w carrageenan and no carob.

A composition of the present invention in the form of a w/w emulsion is contemplated to have the gelling or swelling agent contained in continuous phase and a laxative present in the disperse phase. In the case of a w/w emulsion it is comtemplated that the composition has a suitable stability when stored at a temperature of from 0 to 5° C., i.e. a stability of at least about 12 months. Also, in the case of a w/w emulsion syneresis may take place, but it has not been observed for any of the compositions described herein.

Examples of suitable laxative agents for use in a composition of the invention include bulk-forming laxatives, osmotic laxatives and peristalsis increasing laxatives.

Preferred laxative agents are polyethylene glycol, lactulose, sodium sulfate, sodium phosphate, magnesium sulfate, magnesium citrate, sodium picosulfate, phosphates (e.g. combination of monobasic sodium phosphate and dibasic sodium phosphate), magnesium carbonate, senna extracts, prune extracts, fig extracts, poloxamers, carob gums, cascara, phenolphthalein, castor oil, docusate salts, liquid paraffin, sugar alcohols and bisacodyl.

In the present context, polyethylene glycol (PEG) is preferred, optionally in combination with lactulose. Some of the other laxative agents may be incorporated in minor amounts.

PEG 3350 is preferred, but it is contemplated that PEGs, alone or in combination, with molecular weight in a range of from about 2,000 to about 8,000 are suitable for use. It is also contemplated that the total concentration of PEG(s) is as described herein for PEG 3350. PEG 3350 is an osmotic laxative and works by increasing stool volume through increased hydration and triggering colon motility.

The concentration of the laxative agent(s) in a composition of the invention corresponds to from about 20% to 100% of its therapeutically effective dose. Normally, a GI-cleansing composition is administered in two, four or eight divided doses, one, two or four in the evening before colonoscopy and the other one, two or four 4-6 hours before colonoscopy. Normally, the dose in the so-called “split-regimen” is the same, i.e. 12.5%, 25% or 50% w/w of the therapeutically effective dose is present in a composition of the invention. In the clinical study reported herein, the test persons ingested four portions (á 62.5 g) in the evening and four portions in the morning, each portion was ingested together with 250 mL of water. The split in portions was made to ensure sufficient intake of water. Moreover, the split in portions makes it possible to ingest portions with different taste as described herein.

A therapeutically effective dose for PEG 3350 is in a range of from 100 to 400 g. A therapeutically effective dose for sodium sulfate is in a range of from 5 to 60 g. A therapeutically effective dose for lactulose is in a range of from about 5 to 50 g normally 3 times a day, i.e. a daily dose from about 15 to about 150 g.

When PEG is combined e.g. with lactulose, the same dose of PEG is employed and lactulose is present in a composition of the invention in a concentration of from 40 to about 120 g.

In general, the concentration of a laxative in a composition of the invention is from about 25 to about 80% w/w based in the weight of the total composition. Notably, the concentration of a laxative is from about 30 to about 70% w/w. The total concentration of laxatives in a composition of the invention is in a range of from about 35 to about 70% w/w based on the weight of the total composition, notably from about 40 to about 80% w/w. Specific concentrations are given in the examples herein. Please note that the present of eg a taste-masking agent that may have laxative effect is not included in the above-mentioned concentrations. As seen from the examples herein a composition of the invention may comprise from about 20% to about 50% of a PEG such as PEG 3350 and from about 10% to about 30% w/w of lactulose (as a powder). More specifically, a composition of the invention comprises from about 30% to about 40% w/w of PEG such as PEG 3350 and from about 10% to about 25% w/w of lactulose (as a powder).

As flavor-improving or taste-improving agents, the following may be employed: sugars, honey, pH-adjusting agents, fruit extracts, salt, aetheric oils, artificial elducorants, flavoring agents such as citrus, vanilla, cinnamon, ginger, lemon etc.

It is advantageous to include a taste-improving agent that also imparts laxative properties to the composition. Examples of such taste-improving agents are prune extract, plum extract, fig extract, coffee extract.

A taste-improving agent, which also is a laxative, is normally present in a composition of the invention in a concentration of from about 5 to about 20% w/w such as from about 7 to about 15% w/w of the total weight of the composition. As seen from the examples herein, a concentration of a fruit extract eg prune extract of about 8-9% w/w was found acceptable. The concentrate used in the examples herein had a DEGREE BRIX of 70 (Degrees Brix (symbol ° Bx) is the sugar content of an aqueous solution. One degree Brix is 1 gram of sucrose in 100 grams of solution and represents the strength of the solution as percentage by mass. If the solution contains dissolved solids other than pure sucrose, then the ° Bx only approximates the dissolved solid content. The ° Bx is traditionally used in the wine, sugar, carbonated beverage, fruit juice, and honey industries).

Optionally, a taste-improving agent like eg aetheric oil, flavor agent, fruit extract or artificial elducorants may be present in a composition of the invention. When present, such an agent is present in a concentration of from about 0.02 to about 5% w/w of the total weight of the composition. Thus, in case of eg vanilla, citrus, cinnamon, ginger, lemon etc. the concentration is from about 0.02-2% w/w such as from about 0.05-1% w/w.

Optionally, sugars, honeys or other sweetening agents may be present in a composition of the invention. If present, such an agent is normally present in a concentration of from about 2 to about 10% w/w.

Regarding taste-masking agents or taste-improving agents it may be an advantage to compose the 2-4 compositions that corresponds to one dose of laxative in such a manner that it follows the main tastes of a normal meal, i.e. a starter, main dish and dessert. It could be compositions having taste of lemon, followed by taste of salt and/or bitter taste followed by sweet taste or the like. Other combinations could also be preferred, just ensuring that the patient may choose between tastes so that it is possible to vary the taste to improve compliance.

Other additives may be included in the composition such as e.g. sodium citrate, ascorbic acid, sodium ascorbate or other pH-regulating agents, especially acidic reacting agents to improve taste. The concentration of such additives, if present, is normally in a range of from about 1 to about 5% w/w.

A composition of the invention may also contain one or more electrolytes to prevent electrolyte imbalance. Suitable electrolytes include monovalent, divalent, or multivalent salts. More specifically the salt is selected from inorganic salts including various alkali metal salts, in particular sodium and potassium, metal and/or alkaline earth metal sulfates, chlorides, borates, bromides etc and ionizable organic salts such as citrates, acetates, lactates, fumarates etc.

Notably, the following salts are of interest: calcium sulfate, sodium chloride, potassium sulfate, potassium chloride, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, alkali metal chlorides, sodium fluoride, organic acids such as citric acid, succinic acid, fumaric acid, malic acid, maleic acid, glutaric acid, lactic acid and the like; dihydrogen sodium phosphate, monohydrogen sodium phosphate, disodium hydrogen phosphate, and mixtures thereof.

If present, the concentration of the individual electrolytes is aimed at complying with the Oral Rehydration Solution from WHO, which contains 90 mmol sodium/L and/or 45 mmol potassium)/L, or the current standard reduced-osmolarity ORS, which contains 75 mmol sodium/L, and/or 40 mmol potassium/L.

In general, the concentration range for sodium in a composition of the invention is from 50 to 120 mmol/L, the range for potassium is from 30 to 60 mmol/L, and if present the range for zinc is up to 100 mmol/L (all calculated as the base).

As seen from the examples herein, electrolytes like potassium chloride, sodium citrate and calcium carbonate may be used. Normally, the total concentration is from about 2 to about 7% w/w, notably from about 2 to about 5% w/w.

Some of the electrolytes mentioned above may also be added in order to adjust pH of a composition of the invention. It is contemplated that the taste of a composition of the invention preferably should have a slightly acidic taste. This can be achieved by adding a pH-adjusting agent to the composition, if necessary, or, alternatively, to top the composition of the invention with granules or a powder containing a pH-adjusting agent. Suitable pH-adjusting agents are citric acid, sodium citrate, ascorbic acid, malic acid, maleic acid, tartaric acid, phosphoric acid, hydrochloric acid and combinations thereof.

It is also important to note that the taste will impart a natural thirst and, accordingly, it will be easier for the patient undergoing endoscopy such as colonoscopy to drink the amount of liquid required. Normally, 0.6-1.5 liters must be taken together with the GI-composition. It is contemplated that drinking of carbonized or sparkling water together with the composition makes it easier to ingest the composition and to ensure the emptying of the composition from the stomach to the intestines.

A composition of the invention also contains water. The concentration of water in a composition of the invention is from about 15 to about 60%, such as from 20 to about 60% or from about 25 to about 60% w/w, notably from about 20 to about 30% w/w or from about 30 to about 50% w/w.

As mentioned above, a GI-cleansing composition comprises
i) one or more gelling or swelling agents,
ii) one or more laxative agents,
iii) one of more taste-improving agents, and
iv) water.

Typically, and based on the Examples herein, a GI-cleansing composition comprises
i) from about 1 to about 15% w/w of a gelling or swelling agent,
ii) from about 25 to about 80% w/w of a laxative agent,
iii) from about 1 to about 20% w/w of a taste-improving agent, and
iv) from about 20 to about 60% w/w or from about 25 to about 60% w/w of water, and
v) optionally one or more additives.

Alternative compositions—based on the Examples—comprise:

### Composition A:

i) from about 1 to about 10% w/w of a gelling or swelling agent,
ii) from about 25 to about 80% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition B:

i) from about 2 to about 7% w/w of a gelling or swelling agent,
ii) from about 25 to about 80% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition C:

i) from about 1 to about 15% w/w of a gelling or swelling agent,
ii) from about 30 to about 75% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition D:

i) from about 1 to about 15% w/w of a gelling or swelling agent,
ii) from about 35 to about 70% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition E:

i) from about 1 to about 10% w/w of a gelling or swelling agent,
ii) from about 30 to about 75% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition F:

i) from about 1 to about 10% w/w of a gelling or swelling agent,
ii) from about 35 to about 70% w/w of a laxative agent,
iii) from about 1 to about 20% w/w of a taste-improving agent, and
iv) from about 20 to about 60% w/w or from about 25 to about 60% w/w of water, and
v) optionally one or more additives.

### Composition G:

vi) from about 2 to about 7% w/w of a gelling or swelling agent,
vii) from about 30 to about 75% w/w of a laxative agent,
viii) from about 1 to about 20% w/w of a taste-improving agent, and
ix) from about 20 to about 60% w/w or from about 25 to about 60% w/w of water, and
x) optionally one or more additives.

### Composition H:

vi) from about 2 to about 7% w/w of a gelling or swelling agent,
vii) from about 35 to about 70% w/w of a laxative agent,
viii) from about 1 to about 20% w/w of a taste-improving agent, and
ix) from about 20 to about 60% w/w or from about 25 to about 60% w/w of water, and
x) optionally one or more additives.

### Composition I:

vi) from about 1 to about 10% w/w of a gelling or swelling agent,
vii) from about 25 to about 80% w/w of a laxative agent,
viii) from about 1 to about 20% w/w of a taste-improving agent, and
ix) from about 20 to about 60% w/w or from about 30 to about 50% w/w of water, and
x) optionally one or more additives.

### Composition J:

vi) from about 2 to about 7% w/w of a gelling or swelling agent,
vii) from about 25 to about 80% w/w of a laxative agent,
viii) from about 1 to about 20% w/w of a taste-improving agent, and
ix) from about 20 to about 60% w/w or from about 30 to about 50% w/w of water, and
x) optionally one or more additives.

### Composition K:

vi) from about 1 to about 15% w/w of a gelling or swelling agent,
vii) from about 30 to about 75% w/w of a laxative agent,
viii) from about 1 to about 20% w/w of a taste-improving agent, and
ix) from about 20 to about 60% w/w or from about 30 to about 50% w/w of water, and
x) optionally one or more additives.

### Composition L:

vi) from about 1 to about 15% w/w of a gelling or swelling agent,
vii) from about 35 to about 70% w/w of a laxative agent,
viii) from about 1 to about 20% w/w of a taste-improving agent, and
ix) from about 20 to about 60% w/w or from about 30 to about 50% w/w of water, and
x) optionally one or more additives.

### Composition M:

xi) from about 1 to about 10% w/w of a gelling or swelling agent,
xii) from about 30 to about 75% w/w of a laxative agent,
xiii) from about 1 to about 20% w/w of a taste-improving agent, and
xiv) from about 20 to about 60% w/w or from about 30 to about 50% w/w of water, and
xv) optionally one or more additives.

### Composition N:

xi) from about 1 to about 10% w/w of a gelling or swelling agent,
xii) from about 35 to about 70% w/w of a laxative agent,
xiii) from about 1 to about 20% w/w of a taste-improving agent, and
xiv) from about 20 to about 60% w/w or from about 30 to about 50% w/w of water, and
xv) optionally one or more additives.

### Composition O:

xvi) from about 2 to about 7% w/w of a gelling or swelling agent,
xvii) from about 30 to about 75% w/w of a laxative agent,
xviii) from about 1 to about 20% w/w of a taste-improving agent, and
xix) from about 20 to about 60% w/w or from about 30 to about 50% w/w of water, and
xx) optionally one or more additives.

### Composition P:

xvi) from about 2 to about 7% w/w of a gelling or swelling agent,
xvii) from about 35 to about 70% w/w of a laxative agent,
xviii) from about 1 to about 20% w/w of a taste-improving agent, and
xix) from about 20 to about 60% w/w or from about 30 to about 50% w/w of water, and
xx) optionally one or more additives.

The laxative agent mentioned in ii) above (and in composition Q to X below) does not include the concentration of a taste-improving agent that may have laxative properties as well. If the taste-improving agent is an agent, which also is a laxative, such as those described herein before, then the concentration of such an agent normally is from about 5 to about 20% w/w based on the total weight of the composition.

The one or more additive (also for the compositions below) may be anyone of those mentioned herein.

The content of gelling or swelling agent must be balanced with the content of water in order to obtain a pudding-like structure. Thus, the weight ratio between the amount of gelling or swelling agent and the amount of water in the composition of the invention is typically from about 0.01 to about 0.8 or from about 0.05 to about 0.3. The weight of the content of water is from about 1 to about 100 times greater than the weight of the content of a gelling or swelling agent. As seen from the examples, the content of water is from about 5 to about 15 times greater than the weight of the content of a gelling or swelling agent. However, when carrageenan or a combination of carrageenan and carob is used, then the weight ratio between the total amount of gelling or swelling agent(s) and the amount of water in the composition is typically from about 1:50 to about 1:100. As seen in the examples herein, the content of water in such composition is from about 50 to 100 times greater such as about 55 to 75 times greater than the total content of gelling or swelling agent(s).

A composition of the invention may also be selected from one of the following compositions, wherein water generally is present in a concentration of from about 15% to about 60% w/w, notably from about 17 to about 40% w/w or from about 20 to about 30% w/w based on the total weight of the composition.

### Composition Q:

i) from about 0.1% to about 10% w/w of one or more gelling or swelling agents,
ii) from about 35% to about 70% of one or more laxative agents (when lactulose is used it is calculated as lactulose powder),
iii) from about 0.02% to about 2% w/w one of more taste-improving agents selected from vanillin, citrus, lemon, cinnamon, ginger etc,
iv) from about 2% to about 15% w/w of a fruit extract such as prune concentrate, and
v) optionally from about 1% to about 5% w/w electrolytes
vi) water.

### Composition R:

i) from about 0.1% to about 5% w/w of one or more gelling or swelling agents,
ii) from about 40% to about 60% of one or more laxative agents (when lactulose is used it is calculated as lactulose powder),
iii) from about 0.02% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

### Composition S:

i) from about 0.1% to about 3% w/w of one or more gelling or swelling agents,
ii) from about 45% to about 55% of one or more laxative agents (when lactulose is used it is calculated as lactulose powder),
iii) from about 0.05% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

### Composition T:

i) from about 0.1% to about 2% w/w of one or more gelling or swelling agents,
ii) from about 47% to about 53% of one or more laxative agents (when lactulose is used it is calculated as lactulose powder),
iii) from about 0.05% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

### Composition U:

i) from about 0.1% to about 1% w/w of one or more gelling or swelling agents,
ii) from about 47% to about 53% of one or more laxative agents (when lactulose is used it is calculated as lactulose powder),
iii) from about 0.05% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

### Composition V:

i) from about 0.1% to about 0.5% w/w of one or more gelling or swelling agents,
ii) from about 48% to about 52% of one or more laxative agents (when lactulose is used it is calculated as lactulose powder),
iii) from about 0.05% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

### Composition X:

i) about 0.18% to about 0.4% w/w of one or more gelling or swelling agents,
ii) from about 48% to about 52% of one or more laxative agents (when lactulose is used it is calculated as lactulose powder),
iii) from about 0.05% to about 1% w/w one of more taste-improving agents selected from vanillin, citrus, lemon, cinnamon, ginger etc,
iv) from about 5% to about 15% w/w of a fruit extract such as prune concentrate, and
v) optionally from about 1% to about 3% w/w electrolytes
vi) water.

In the above-mentioned compositions, the individual ingredients may be selected among those mentioned herein before.

A composition of the present invention may be used in the treatment or alleviation of constipation, in the cosmetic treatment of overweight, in the medical treatment of overweight or obesity.

Moreover, as the main focus, a composition of the invention may be used in endoscopy, notably in colonoscopy.

### LEGENDS TO FIGURES

FIG. 1 show the structure of the three samples from example

FIGS. 2-4 show images of the samples with 100× magnification;

FIGS. 2-3: Note the globular particles (dispersed phase) and their size

FIG. 4 is the batch used in the clinical testing

FIGS. 5A-C show the microscopic structure as the two phases are mixed illustrating the spontaneous formation of the globular particles at the interphase between the 2 phases. The 3 pictures (FIG. 5A-C) were taken at 30 seconds intervals

FIG. 6-7 show the results of the rheological measurements

FIG. 8 shows the result of the clinical study on a BBP scale

The invention is further illustrated in the following, non-limiting examples

### EXAMPLES

### Example 1

### A Composition According to the Invention to be Administered Twice

The composition below is intended to be administered the evening before and/or the morning before colonoscopy. Thus, it contains 50% of the active ingredients. Compositions may also be made eg where one of the compositions contains 40% of the total dose and the other contains 60% of the total dose. Other percentage distributions may also be employed and a person skilled in the art will know how to adjust the ingredients in order to arrive at such compositions. One of two doses (One “portion” evening OR morning)

A. General Composition to which Additives and Adjusting Agents May be Added:

| Ingredient | Amount (g) | |
|---|---|---|
| 1. Laxative (eg Macrogol 3350) | 100 | |
| 2. Water | 100 | |
| 3. Taste-improving agent (laxative properties) | 25 | g |
| 4. Gelling or swelling agent | 7.5-15 | g |

step a. mix 1, 2, 3, and 4, and blend.

step b. bring to boil.

step c. Pour into container and allow the mix to set.

Amount before step b=232.5-240 g Amount after step c=about 220 g (about 10% reduction in weight)

B. Specific Composition Containing Gellan as a Gelling or Swelling Agent:

| Ingredient (Gellan) | Amount (g) |
|---|---|
| 1. Macrogol 3350 | 100 |
| 2. Lactulose | 40 |
| 3. Potassium chloride | 1 |
| 4. Sodium chloride | 2 |
| 5. Sodium citrate | 7.5 |
| 6. Water | 100 |
| 7. Prune extract | 25 |
| 8. Anisyl alcohol | 0.5 |
| 9. Gellan | 7.5-15 |

step a. mix 1, 2, 3, 4, 5, 6, 7, 8 ad 9 and blend.

step b. bring to boil.

step c. Pour into container and allow the mix to set.

Amount before step b=0.275 kilograms

Amount after step c=0.251 kilograms

C. Specific Composition Containing Agar as a Gelling or Swelling Agent

| Ingredient (Agar) | Amount (g) |
|---|---|
| Macrogol 3350 | 100 |
| Lactulose | 40 |
| Potassium chloride | 1 |
| Sodium chloride | 2 |
| Sodium citrate | 7.5 |
| 6. Water | 100 |
| Prune extract | 25 |
| Anisyl alcohol | 0.5 |
| agar powder | 7.5-15 |

step a. mix 1, 2, 3, 4, 5, 6, 7, 8 ad 9 and blend.

step b. bring to boil.

step c. Pour into container and allow the mix to set.

Amount before step b=0.275 kilograms

Amount after step c=0.254 kilograms

D. Specific Composition Containing Pectin as a Gelling or Swelling Agent

| Ingredient (Pectin) | Amount (g) |
|---|---|
| Macrogol 3350 | 100 |
| Lactulose | 40 |
| Potassium chloride | 1 |
| Sodium chloride | 2 |
| Prune extract | 25 |
| 6. Water | 100 |
| Anisyl alcohol | 0.5 |
| Pectin | 7.5 |

step a. mix 1, 2, 3, 4, 5, 6, 7, 8 ad 9 and blend.

step b. bring to boil.

step c. Pour into container and allow the mix to set.

Amount before step b=0.275 kilograms

Amount after step c=0.248 kilograms

### Example 2

### A Composition According to the Invention Including Crème Sauce

The compositions as described in Example 1 are made, but only using 80% of the gelling and swelling agent. This result in that the gelling or swelling agent exhibits syneresis, i.e. some of the liquid is not totally sorbed by the gelling or swelling agent and a crème sauce like-structure appears.

Alternatively, a crème sauce may be prepared by preparing the compositions of Example 1, but leaving out the gelling or swelling agent. The dose of the crème sauce (in a twice split dose regimen) may be from 10 to 50% w/w of the weight of the sauce obtained.

### Example 3

### A Novel Bowel Cleansing Pudding Formulation

### Formulation

Content for one 250 g portion of pudding

| Components | Weight (g) |
|---|---|
| Polyethylene glycol 3350 (PEG, Macrogol, | 100 |
| Pharma quality from DOW) | |
| Lactulose Powder | 40 |
| Potassium chloride | 1.12 |
| Sodium citrate-dihydrate | 5.17 |
| Calcium carbonate | 1 |
| MiliQ-Water | 100 ml |
| Prune concentrate (Rynkeby) | 25 |
| Vanillin | 0.2 |
| K-dominated-Carrageenan (GENU LACTA) | 0.5 |
| Carob (GENU GUM) | 0.5 |

Apparatus used in preparation of the pudding

Apparatus

| | |
|---|---|
| Magnetic stirrers | 250 mL Erlenmeyer flask |
| Water bath (50° C.) | Metal bowl x2 |
| Food mixer with whisk attachment | Disposable weighing boats |
| Magnetic stirrer hot plate x2 | Large spoons |
| Weighing scales | Glass bowl/beaker as final vessel for |
| | pudding |
| Thermometers | Small spoons and spatulas |

### Procedure:

To produce a 250 g portion of pudding. Phase 1 and phase 2 must be produced separately before mixing.

### Phase 1:

1. Warm the metal bowl of the food mixer in a water bath set to 50° C. for 10-20 minutes.
2. Weigh out and combine 100 g PEG, 1.12 g potassium chloride, 5.17 g sodium citrate, 1 g calcium carbonate, and 1 g vanillin and set aside.
3. Heat the 25 g prune concentrate with 20 ml of warm water using the magnetic stirrer hot plate to around 40° C. Once it has come to temperature, add the prune and water mixture to the pre heated food mixer.
4. Then slowly add the powders to the food mixer (containing the prune and water), and mix on a high speed until fully combined.
5. Transfer the metal bowl of the food mixer back to the water bath, to heat to 40° C. Keep stirring this regularly using the whisk attachment from the food mixer, until a smooth, homogenous phase is formed.

### Phase 2:

1. Heat 60 ml 66.7% Lactulose Solution and 60 ml water to 95° C. using a magnetic stirrer hot plate and electronic contact thermometer. Use a tall, thin beaker for this in order to achieve an effective vortex. Cover beaker with aluminium foil to speed up the process.
2. Meanwhile combine 0.5 g carrageenan, 0.5 g carob (Locust Bean Gum.
3. Once the water has come to temperature, add the polymers at a steady speed, aiming for the powder to hit the side of the vortex (to ensure rapid dispersion).
4. Wait until the solution becomes clear with no particulates, while keeping at 90° C. under agitation.

Combining Phase 1 and 2
1. Once phase 1 is completely dissolved (within the water bath) place the metal bowl back on the food mixer and start to mix on a fast speed.
2. Let phase 2 cool to around 60° C., then add slowly into the food mixer, ensuring that the stream of liquid hits as close to the side of the metal bowl as possible.
3. Continue to mix on a high speed until the two phases are fully combined, and the colour and consistency start to change.
4. Wait until the formulation reaches 25° C., still continually mixing, before pouring into glass beakers.

### Example 4

### Basic Formulation and Procedure

Content for one 250 g portion of pudding

| Component | Weight (g) |
|---|---|
| Polyethylene glycol 3350 (PEG, Macrogol, | 100 |
| Pharma quality from DOW) | |
| Lactulose (NN) | 40 |
| Potassium chloride | 1.12 |
| Sodium citrate-dihydrate | 5.17 |
| Calcium carbonate | 1 |
| MiliQ-Water | 100 |
| Prune concentrate (Rynkeby) | 25 |
| Vanillin extender flavour (Firmenich) | 0.5 |
| κ-Carrageenan (kappa carrageenan) | 1.5 |

### Procedure:

To produce a 250 g portion of pudding. Phase 1 and phase 2 must be produced separately before mixing.

### Phase 1:

1. Weigh 100 g PEG and place it in an Erlenmeyer flask. Add 50 mL water to the flask. Heat using a heat plate to 33° C. while stirring with a magnetic bar until complete dissolution (the solution will be visibly clear).

### Phase 2:

1. (a) Weigh off, and mix together in a bowl; 40 g lactulose, 1.12 g potassium chloride, 5.17 g sodium citrate, 1 g calcium carbonate, 25 g prune concentrate and 0.5 g vanillin extender flavour.
2. (b) Weigh off 1.5 g κ-Carrageenan and sprinkle it, under vigorous stirring, onto 50 mL water in the plastic cup (1 L) of an immersion blender. Pour the solution in a bowl and heat it to 90° C. Once the temperature reaches 90° C., let the solution cool to 60° C. at room temperature.
3. When the temperature of (b) reaches 60° C., pour it into (a) and mix together with a large the immersion blender (this seems to give a more stable overall result compared to mixing with a spoon of whisk). Place the solution in the water bath for a couple of minutes to achieve a more homogeneous blend.

### Mixing of Phase 1 and Phase 2:

1. Mix phase 1 and Phase 2 at room temperature by pouring phase 1 in phase 2 and mixing with a large spoon or a whisk (not an immersion blender as this results in a weaker emulsion).
2. Pour the formulation into the glass bowl, let the air bubbles resolve and cover it with Parafilm. Place the serving bowl with the formulation in a −18° C. freezer overnight and then refrigerate it (at 5° C.). Note: After freezing overnight (at least 8 hours), it is very important to immediately transfer the formulation from the freezer to a refrigerator, as it has an influence on the stability of the formulation.

### Example 5

### Taste-Masking

### Vanilla Flavour

The taste/flavour of the pudding was first to be adjusted by varying the concentrations and forms of vanilla flavouring as seen below in Table 1, The percentages were chosen as this almost represents a logarithmic scale. The pudding was produced per procedure in Example 4 with only the vanilla element changing. The results were assessed by a taste panel.

TABLE 1

| Vanilla Extender Flavour | | |
|---|---|---|
| (VEG) | Vanillin | Ethyl-Vanillin |
| 0.15 g | 0.15 g | 0.15 g |
| 0.5 g | 0.5 g | 0.5 g |
| 1.5 g | 1.5 g | 1.5 g |

Vanillin was found to be superior. The concentration was further adjusted throughout the investigation, and assessed again using a taste panel, as seen in Table 2 and 3:

TABLE 2

| | 0.05 g | 0.1 g | 0.2 g | 0.3 g | 0.4 g | 0.5 g |
|---|---|---|---|---|---|---|
| Initial adjustments | 0.1% | 0.2% | 0.4% | 0.6% | 0.8% | 1% |
| (Concentration in | | | | | | |
| 50 g of pudding) | | | | | | |

TABLE 3

| | 0.2 g | 0.6 g | 1 g | 1.5 g |
|---|---|---|---|---|
| Further adjustments in the reduction | 0.08% | 0.24% | 0.4% | 1% |
| in vanillin | | | | |
| (concentration in 250 g of pudding) | | | | |

The vanillin (Sigma Aldrich) was the preferred flavour according to the taste panel. See Table 4 below regarding the rankings of the pudding:

TABLE 4

| Ranking | Flavour used per 50 g pudding |
|---|---|
| 1^{st} place | 0.3 g Vanillin - creamy |
| Joint 2^{nd} place | 0.03 g Vanilla Extender Flavour (VEF), |
| | 0.3 g VEF |
| 3^{rd} place | 0.1 g Vanillin |
| 4^{th} place | 0.3 g Ethyl Vanillin |
| 5^{th} place | 0.1 g Ethyl Vanillin |
| 6^{th} place | 0.1 g VEF |
| 7^{th} place | 0.03 g Vanillin |
| 8^{th} place | 0.03 g Ethyl Vanillin |
| Joint 1^{st} place | 0.2 g vanilli., 0.3 g vanillin |
| Joint 2^{nd} place | 0.05 g Vanillin, 0.5 g Vanillin |
| 3^{rd} place | 0.4 g Vanillin |
| 4^{th} place | 0.1 g Vanillin |

Due to the results of the taste panel, at first 0.3 g Vanillin in 50 g (0.6% w/w) was selected for use in the clinical testing. However, while making the protocol for the clinical trial batch at HB Medical, the puddings were tasted again and it was found that the flavour of vanilla gets too strong after a couple of mouthfuls of pudding. Hence, further adjustments were made. 0.2 g Vanillin (0.08%) was used as a starting point for the final vanillin concentration in the clinical test studies. Due to the content of vanillin, it may be considered to add an antioxidant to avoid any oxidation of vanillin to vanillic acid.

### Example 6

### Carrageenan Concentration

In an attempt to remove the freezing process from the procedure, the k-dominated-carrageenan concentrations were increased (see Table 5 and 6), but it was found to be impossible to disperse and dissolve in cold water. A hand-blend was used to initially disperse the carrageenan in cold water, then dissolve it in hot (90° C. water), as per Example 4. Using the vortex method (as explained in the Handbook of Pharmaceutical Excipients (Rowe, 2006)) and with the water at 90° C., the carrageenan dispersed and dissolved simultaneously, and agitation was continued until full hydration is achieved. Also dry-blending the carrageenan with the lactulose acted to adequately disperse the polymer.

TABLE 5

| | Carrageenan | | |
|---|---|---|---|
| | concentration | | |
| | 0.6 g | 1.2 g | 2.4 g |
| Initial variations to | 0.6% | 1.2% | 2.4% |
| carrageenan (in | | | |
| 100 g pudding) | | | |

TABLE 6

| | Carrageenan concentration | | | | |
|---|---|---|---|---|---|
| | 0.3 g | 0.4 g | 0.6 g | 0.8 g | 1 g |
| Further adjustments, | 0.6% | 0.8% | 1.2% | 1.6% | 2% |
| using 50 g | | | | | |
| pudding sample | | | | | |

Changed to vortex method with a 3 g in 250 g concentration (1.2%).

Both κ- and iota carrageenan were used.

The results showed that better results were obtained if the carrageenan-carob phase to was heated to almost the boiling point, heating the PEG phase to +/−50° C. under frequent stirring for a time long enough to form an homogenous mixture and mixing the phases at the same temperature, 33° C., for both. Then freezing overnight and transferring to a refrigerator (+5)

### Procedure Adjustments

The main focus was to vary the production temperatures (the examples found in Table 7) to improve the gelling of the pudding and to avoid a step of cooling. The type of carrageenan used was also changed from using the k-dominated carrageenan to iota-carrageenan, using the same concentrations (1.5 g in a 250 g batch of pudding). All adjusted formulations were stored at freezer, fridge and room temperature, to see if the pudding ‘set’ without the need for the freezer, and to assess the texture.

TABLE 7

| Temperature | | | |
|---|---|---|---|
| adjustments | Phase 1 | Phase 2 | Mixing |
| Adjustment 1 | Increase temperature | Keep temperature at | Mix under efficient stirring |
| | to approximately | 60° C. | (at 60° C.) and fill immediately |
| | 60° C. | | into glass bowls |
| Adjustment 2 | Keep temperature | Mix phase 2 (b) at | Immediately after phase |
| | at 33° C. (as | 90° C. with the phase 2 | 2 (a) and (b) are combined. |
| | original method) | (a). | Mix this hot solution |
| | | | with phase 1. |
| Adjustment | Keep at 33° C. | Keep phase 2 (a) at | Mix phase 2 (b) with the |
| 3 (cold process) | | room temperature, | other phase under efficient |
| | | and mix with phase 1. | mixing. |
| | | Make phase 2 (b) as | |
| | | usual but cool to room | |
| | | temperature. | |

From the table above, Phase 1: PEG and water. Phase 2 (a): lactulose, potassium chloride, sodium citrate, calcium carbonate, prune concentrate and vanillin extender flavour. Phase 2 (b): κ-dominated carrageenan and water.

Tests were also made where the mixing technique was also reversed so instead the polymer phase (carrageenan+carob phase) was added to the PEG phase in the aim to get a different gel consistency. The samples were then left to set directly in the refrigerator or in the freezer overnight and then transferred to the refrigerator. The samples which were in the freezer overnight appeared more homogeneous and, compared to those which were all the time in the refrigerator displayed a lesser tendency to separate.

### Example 7

### Addition of Carob

Carob (LBG) was added to the formulation to strengthen the gel, the aim was to stabilise the w/w emulsion using this method. Equal quantities of carrageenan to carob were tested, originally 1.5 g, but reduced eventually to 0.5 g. The carob was added to the carrageenan and lactulose phase, so all the polymers were together, and so the lactulose could aid dispersion. Below details the carob adjustment made over time. The batches were stored in the freezer, refrigerator and at room temperature and then compared.

| Carrageenan concentration | |
|---|---|
| (g) | Carob concentration (g) |
| In 250 g pudding | In 250 g pudding |
| 1.5 g | 1.5 g |
| 1.2 g | 0.8 g |
| 0.5 g | 0.5 g |
| 0.5 g | 0.5 g |
| 0.5 g | 0.3 g |

The addition of carob improved the mouthfeel of the pudding, and seemed to give a smoother texture. The original 1.5 g carrageenan and 1.5 g carob set to a firm gel in the refrigerator but the high concentration of gelling agents meant that even with the vortex method, the polymers were difficult to disperse and dissolve. It was found that even with the lower concentrations, the formulation still set in the refrigerator; aided by the reversal of the mixing technique. Where the carob allowed the pudding to set in the refrigerator, the carrageenan concentration was able to be reduced; this made the dispersion of the polymers much more simple, which was a benefit when it came to upscaling.

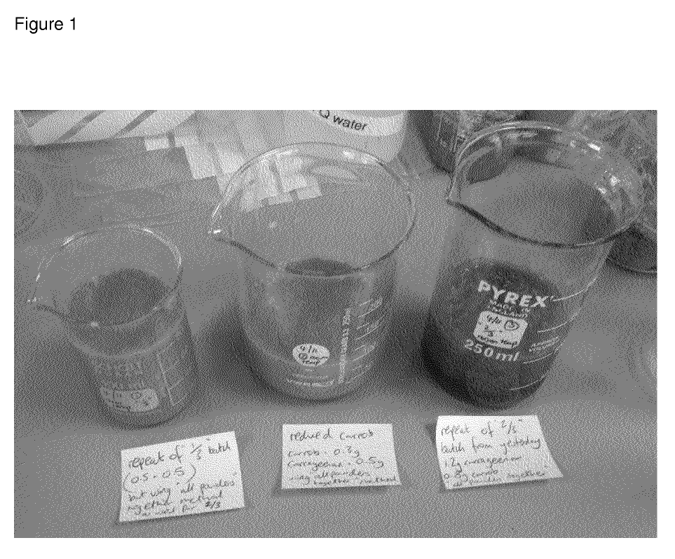

The formulations with the carob nearly set at room temperature with the ‘repeat of ⅓ batch’ from FIG. 1, being very close to perfectly setting.

### Example 8

### Microscopic Assessment

A light microscope—a Leica DM750 with the Leica ICC50 HD Digital Camera Module, was used to visualize the w/w emulsion. A tiny sample of the finished pudding was loaded onto a microscope slide and images were taken on both the 40× band the 100× magnification settings using the LAS V4.1 computer system. Comparisons were made between the images containing carob, and those without carob as well as different samples with varying storage temperatures. The final sample made at HB medical was also imaged to assess the variation when scaling up production.

To further investigate the water-in-water emulsion, the two separate phases where mixed together under the microscope to observe the formation of the emulsion as a microscopic level. The two phases were produced per procedure (Example 3), and left to set in the freezer overnight. The PEG phase was added first to the microscope slide and a cover slip was placed on top. Then a drop of the carrageenan phase was placed next to the cover slip and left to mix with the PEG via capillary action. A Nikon Eclipse Ti microscope was used for this as it gave a better depth of field. Images were taken at various points to show the development of the emulsion droplets.

A phase diagram of PEG was obtained using the DSC. 15 microliters of PEG suspension was used. Started at 5° C., increasing up to 80° C., with an increase of 5° C. every minute.

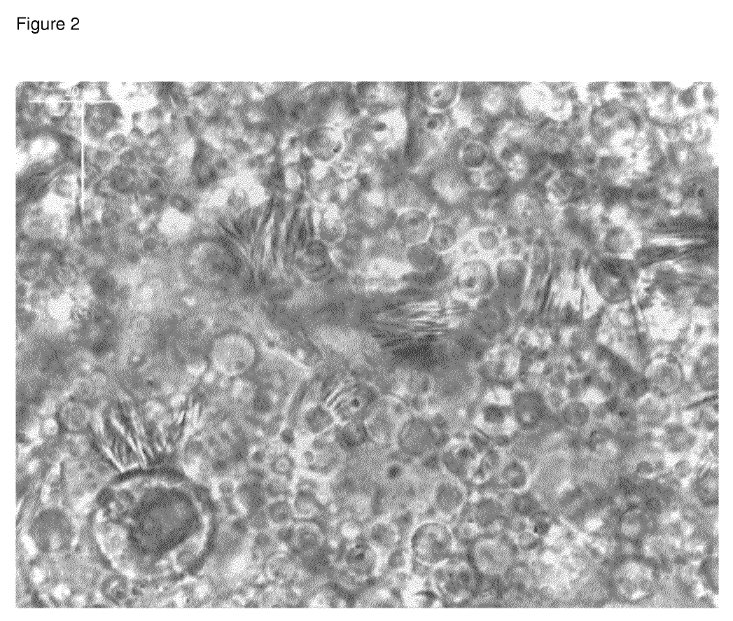

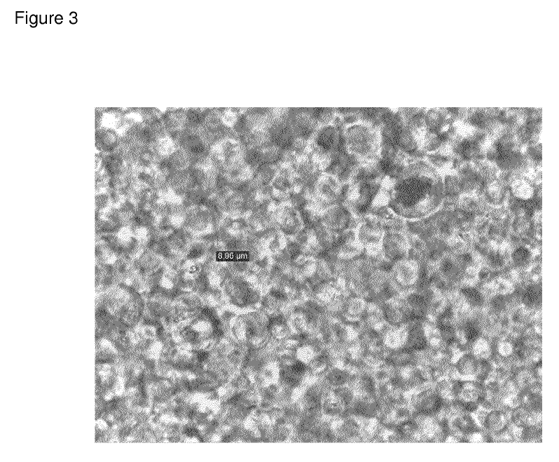

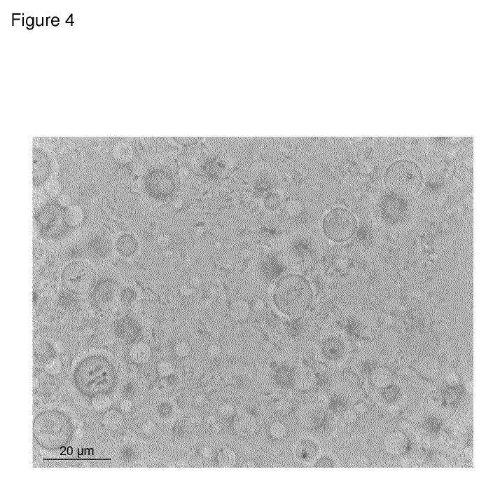

### Microscopic Structures—FIGS. 2, 3, 4, 5A-C

FIGS. 2, 3 and 4 support the initial concept of formation of a water-in-water emulsion, with droplets of an aqueous dispersed phase within an aqueous continuous phase and it is suggested by the images that these phases are immiscible when mixed together. There is no oil or hydrophobic components within the formulation so a typical oil-in-water emulsion cannot be formed. Much less is known about a water-in-water emulsion compared to the typical oil-in-water so it is difficult to verify. However, based on the photos there is a clear indication that two phases are formed The kinetic stability of the emulsion may be difficult to control as surfactants do not adsorb onto the w/w interface (hence surfactants cannot be used as an interphase between the two hydrophilic phases). One way of stabilisation is by gelling one or both of the phases to keeping the two phases dispersed (Nguyen et al., 2013). In the pudding formulation the carrageenan phase is acting as the gelling phase, which could explain why the formulation stays in the water-in-water emulsion (if the gel is set quickly enough) and doesn't separate out into two distinct phase.

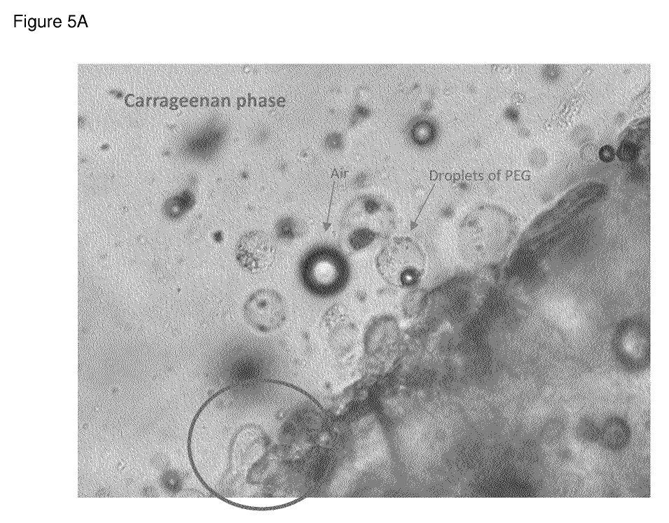

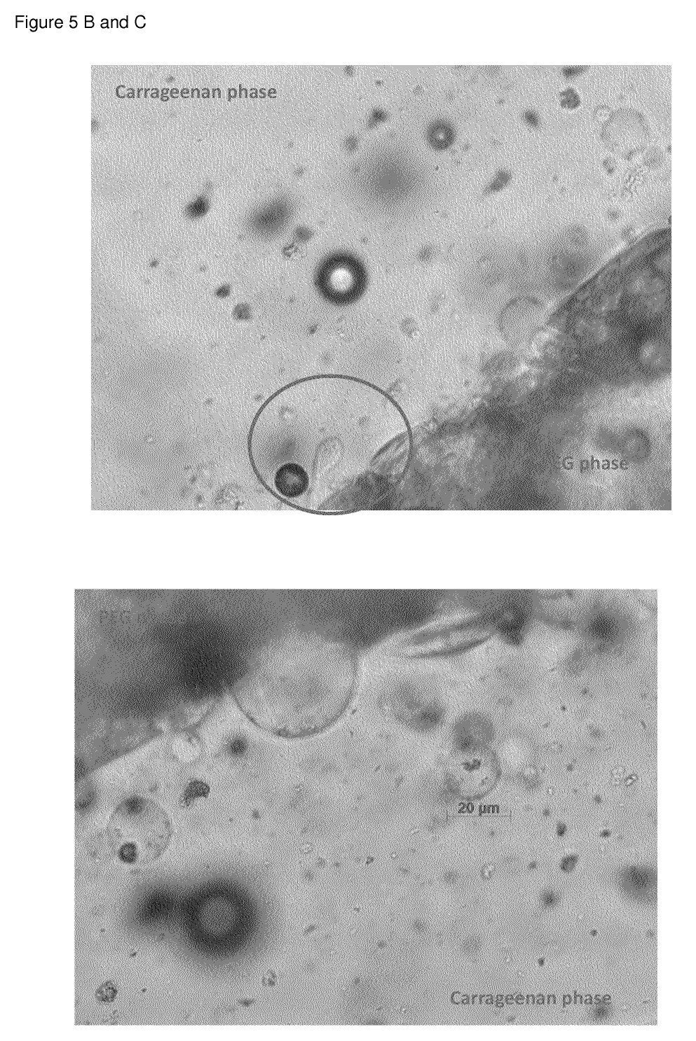

FIGS. 5A-C show the microscopic structure as the two phases are mixed illustrating the spontaneous formation of the globular particles at the interphase between the 2 phases. The 3 pictures (FIG. 5A-C) were taken at 30 seconds intervals

### Example 6

### Rheological Studies

Finally, the rheology of the pudding was studies to compare the stiffness/hardness of the gel of the pudding containing carob vs the one without, and to test the sample for the clinical trial. The rheological measurements were performed on a controlled stress rheometer (AR-G2 Rheometer). Small deformations measurements were obtained using a plate with diameter of 40 cm with an evaporation protection cap. The gap was adjusted to 400 μm (in order to allow for an adequate sample of my pudding to be tested) and the mode was changed to stiff bearing mode. The rheological measurements were performed at 5° C. Frequency and stress sweeps were set up using an oscillation stress of 0.8 Pa (as a stress sweep was used to determine the linear viscoelastic region, and at 0.8 Pa the gel did not break (Malvern.Instruments.Ltd, 2005)). The number of tests run per sample were 3-4. The data was recorded in the Data Analysis programme for the AR-G2 Rheometer and analysed and stored in Microsoft Excel.

### Rheology Results

Small deformation rheology was carried out on the 3 carrageenan gels to measure and compare the elastic modulus G′.

Sample 1 was a standard pudding formulation, as in Example 2 containing 0.2% carob (and 0.2% κ-dominated carrageenan); sample 3 was a pudding produced from the older method, that did not contain carob but contained 0.6% carrageenan as in example 4; sample 2 was from the final clinical trial batch, produced with 0.2% carob and 0.2% carrageenan but using the adjusted method, as in example 2, for upscaling.

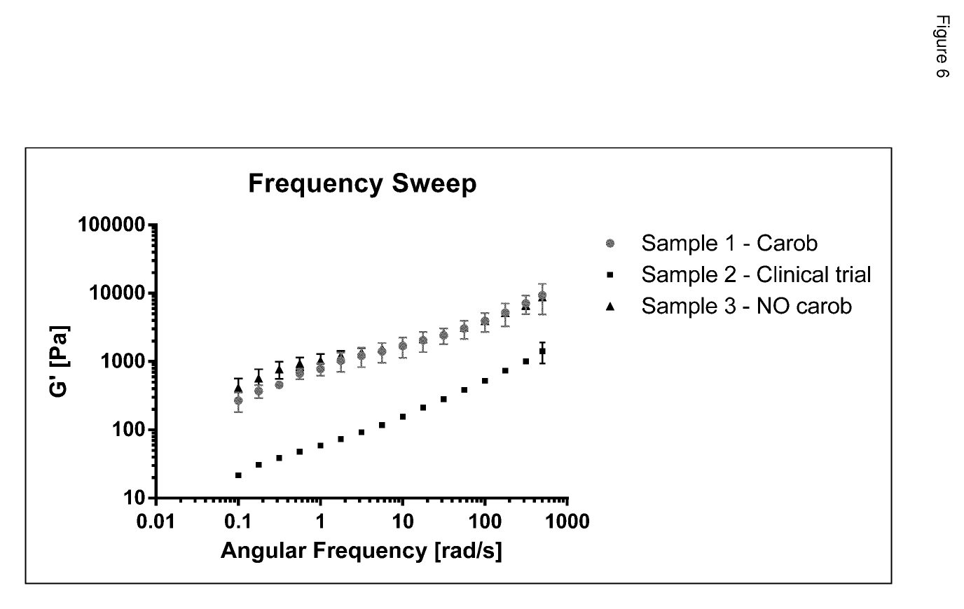

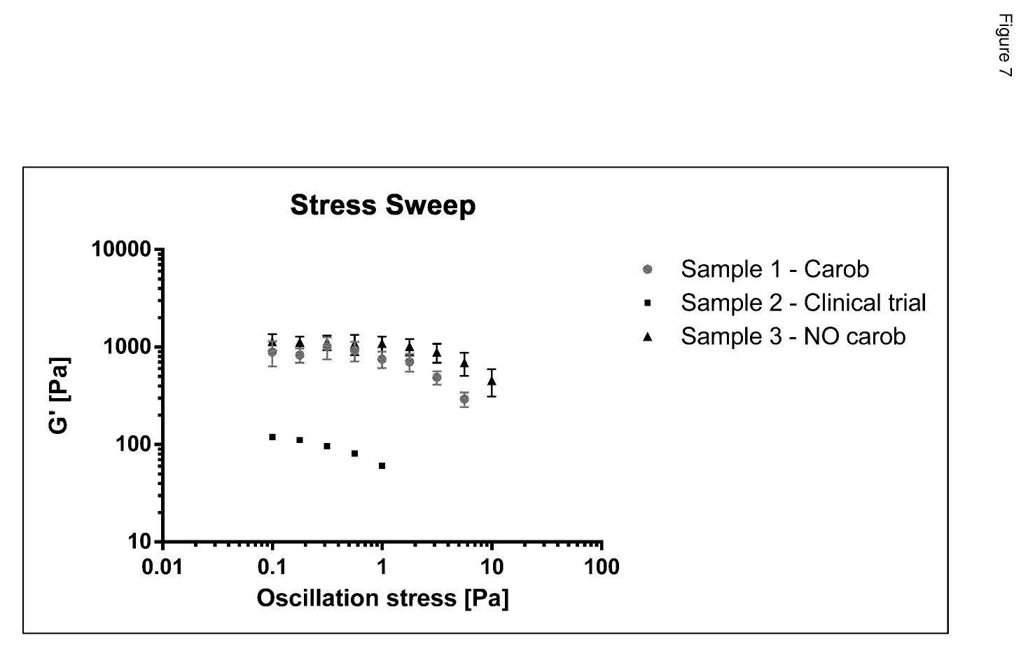

The results of the frequency sweep and stress sweep are shown in FIGS. 6 and 7. The frequency sweep is used to assess the stiffness/hardness of the gel over time and the stress sweep measures at what stress (Pa) the gel breaks at. From the frequency sweep, sample 2 pudding is shown to be softer and less brittle than the others. It likely to deform at a lower stress value. Samples 1 and 3 are confirmed to be a harder gel, and more brittle. They will likely deform at a higher stress value compared to sample 2. A one-way ANOVA was completed, including a Tukey's multiple comparisons test to assess the significance of the differences, as shown in FIG. 7. The p-values verify that there is no significant difference between the hardness of gel with and without carob (between sample 1 and 3), however sample 2 is significantly different.

Sample 2 is measured as being a softer gel, as the gelling network within the sample differs from the other samples.

Interestingly, sample 1 and 3 were not statistically different regarding both the hardness of the gel and the force at which the gel breaks despite one containing carob and one containing a significantly higher concentration of carrageenan. There appears to be no difference between 0.2% carrageenan PLUS 0.2% carob versus 0.6% carrageenan. This suggests that the carob acts to strengthen the gel more than just carrageenan alone.

### Example 9

### Patient Use of the Pudding

The 273.49 g portion of the pudding (that equates to the standard 100 g dose of PEG) should be consumed gradually over a 1-hour period, while drinking at least 1 L of cold water over this time. For example, one quarter of the pudding (roughly 68 g) every 15 minutes with 1 glass of water.

Timetable for initial clinical trial with bowel cleansing pudding:

| Time | Activity |
|---|---|
| 12.00 - day before | Low Residue Meal (no more food allowed |
| | after this) |
| 18.00 | Stomach fully emptied |
| 22.00 | Pudding formulation 4 × 15 mins |
| 6.00 am - day of examination | Pudding formulation 4 × 15 mins |
| 8.00 | Patient fasts - no liquids |
| 10.00 | Colonoscopy Examination |

### (Preliminary) Patient Instructions:

Start the pre-colonoscopy preparation the day before your colonoscopy, with a ‘low residue’ meal at lunchtime—12.00 μm (see below for more information).
From the moment you start the preparation, only clear liquids can be drunk (and nothing coloured red or purple), and no food can be consumed after your ‘no residual’ meal at lunchtime.
Begin the bowel cleansing formulation at 22.00. Take one pot of the pudding (˜68 g) out of the fridge and consume with one glassful of water (250 ml). It is important that all 250 ml of water is consumed. After 15 minutes, so at 22.15, take another pot of pudding out of the fridge and consume this slowly with one glassful of water (250 ml). Continue this process, repeating every 15 minutes, until you have eaten all 4 pots (finishing at 23.00).
Ideally the liquid taken with the pudding is 250 ml of cold mineral water, however the water can be substituted for another clear, cold liquid (but no cola and nothing carbonated).
Repeat this process at 6.00 am the day of your colonoscopy, consuming all 4 pots of pudding slowly at intervals of 15 minutes (finishing at 7.00), again consuming each pot with 250 ml of (ideally) cold mineral water.
From 8.00, no further liquids can be consumed.
10.00 colonoscopy procedure begins.

‘Low residue’ meal is a meal that is low in fibre and other undigested materials to minimise the production of solid waste in your colon and rectum. Examples include: Skinless chicken, fish, eggs, white bread, pasta, white rice, canned or cooked vegetables (without skin or seeds), broth-based soups, honey, jelly (McCray and Balaban, 2007). Unfortunately, your bowel preparation will likely be unpleasant. However, if you do it correctly and follow all instructions, then the colonoscopy will be able to be completed fully and successfully. If your bowel cleansing is not adequate (i.e. there is still solid and liquid stool matter in the bowel), then the procedure will have to be repeated, causing further discomfort. It is in your best interests that you follow all instructions closely.

### Example 10

Clinical Pilot Study with a Composition as Described in Example 3

This pilot study was aimed at evaluating the effect of a new formulation type of bowel preparation before colonoscopy.

At present, most bowel preparations consist of medium to large volume intake of a solution of Poly-Ethylene-Glycol with various salts or more concentrated low volume intake of the same compounds followed by large quantities of clear fluids. Many patients find the taste of these cleansing remedies somewhat problematic and, thus, difficult to drink.

The new edible formulation of cleansing product according to the present invention is an attempt to alleviate the taste, volume, as well as the texture of the cleansing solution.

### Material and Methods:

Ten healthy male volunteers (age 22-49 years) received the preparation. We maintained a standard split dose regimen with no food intake starting 24 hours prior to colonoscopy, but with an intake of clear fluid of at least 250 mL per hour. Approximately, 4 hours after the final intake of the cleansing product a colonoscopy was performed. The quality of cleansing was estimated using the Boston Bowel Preparation Scale (BBPS).

### Results

The volunteers had cleansing BBPS scores at seven or above.

### Conclusion

It is possible to cleanse the bowel with our new product sufficiently for performance of screening colonoscopy.

### Keywords

Boston Bowel Preparation Scale (BBPS), Bowel Preparation, Cleansing, Colonoscopy

### Introduction

Colonoscopy is the preferred procedure for detecting pathology in the large bowel. The validity of the procedure depends upon the degree of cleansing.

Several factors influence the degree of cleansing such as the volume of the preparation, the active agents of the preparation, compliance of the patients, etc. (1).

We have in this context chosen to try to augment the probability of compliance by changing the taste and texture of the cleansing product. In our edible product, the cleansing agents are embedded in a gel with a creamy consistency and the taste has been improved by addition of vanilla and prune extracts. Furthermore, powdered ascorbic acid was supplied for optional sprinkling on top of the product.

The quality of the product rely on the results as judged by colonoscopy, we therefore as a first attempt have given our preparation to volunteers and in the following, we describe our findings.

### Methods and Materials

A batch of cleansing preparation was manufactured (130 portions of 68.2 gram, containing Polyethylene Glycol 3350, Lactulose powder, Potassium Chloride, Sodium Citrate-dihydrate, Calcium Carbonate, Vanillin, Carragenan, Carob, and Prune concentrate. Manufacturer: HB-medical). Each cleansing procedure consisted of a 24-hour fasting period with unlimited access to clear fluids with a minimal intake of 250 mL/hour when awake. At 8:00 pm the evening before the procedure, the volunteers ingested the first four portions of the preparation with a time space of 15 minutes between each portion. The next four portions were taken in the same sequence on the day of the examination starting four hours prior to the procedure. Each cleansing, thus, comprised of eight portions of the preparation in total.

Four hours after the last intake of the product a colonoscopy was performed. All endoscopies were performed by a single endoscopist, who had performed more than 5000 endoscopies. The quality of the cleansing was evaluated by using the Boston Bowel Preparation Scale (2). The endoscopist followed the guidelines put forward in the instructional video available at the site of the Boston University, School of Medicine. Colonoscopies were performed in the Endoscopy unit at the Zeeland University Hospital using Olympus Exera 3 CF-HQ190L colonoscopes with a UPD-device.

Ten healthy male volunteers (age 22 to 49 years) were included.

All volunteers were offered sedation with Fentanyl 50 μg i.v. supplemented with Midazolam 2 mg i.v.

### Results

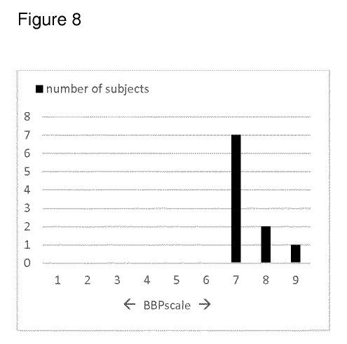

All the included volunteers had followed the instructions for cleansing. When asked three volunteers reported slight bloatedness after the first four portions of the product, otherwise no unpleasant effects were reported. They all had a complete colonoscopy performed without side effects or complications. No pathological findings were seen. The results of the Boston Bowel Preparation Scale are shown in FIG. 8.

### Conclusion

This small pilot study shows that it is possible to cleanse the large bowel with our new edible preparation and the quality of the cleansing seems appropriate for performing screening colonoscopy. This study does not give any clues as to whether patients may prefer our product to other preparations, but future investigations should be performed to show whether our product is a reasonable alternative to already existing cleansing products.

### REFERENCES

1. John R. Saltzman et al. Bowel preparation before colonoscopy. Gastrointest Endosc 2015; 81, 4:781-94 (Guideline from the American Society of Gastrointestinal Endoscopy)
2. Edwin J. Lai et al. The Boston bowel preparation scale: a valid and reliable instrument for colonoscopy-oriented research. Gastrointest Endosc 2009; 69:620-5.

1-26. (canceled)

27. A GI-cleansing composition comprising:
(i) one or more gelling or swelling agents,
(ii) one or more laxative agents,
(iii) one or more taste-improving agents, and
(iv) water.

28. A GI-cleansing composition, wherein from about 100 to about 500 mL of the composition provides one dose of the one or more laxative agents.

29. A GI-cleansing composition according to claim 27, wherein the concentration of the gelling or swelling agent is from about 0.1 to about 15% w/w of the total weight of the composition.

30. A GI-cleansing composition according to claim 27, wherein the concentration of the gelling or swelling agent is from about 2 to about 7% w/w of the total weight of the composition.

31. A GI-cleansing composition according to claim 27, wherein the concentration of the gelling or swelling agent is from about 0.1 to about 5% w/w of the total weight of the composition.

32. A GI-cleansing composition according to claim 27, wherein the concentration of the laxative agent is from about 25 to about 80% w/w of the total weight of the composition.

33. A GI-cleansing composition according to claim 27, wherein the concentration of the laxative agent is from about 35 to about 70% w/w of the total weight of the composition.

34. A GI-cleansing composition according to claim 27, wherein the taste-improving agent is selected from fruit extracts, vanillin, citrus, lemon, cinnamon, ginger, and mixtures thereof.

35. A GI-cleansing composition according to claim 27, wherein the concentration of the taste-improving agent is from about 0.02 to about 20% w/w of the total weight of the composition.

36. A GI-cleansing composition according to claim 27, wherein the taste-masking agent is a fruit extract and the concentration thereof in the composition is from about 1 to about 20% w/w of the composition.

37. A GI-cleansing composition according to claim 27, comprising vanillin, citrus, lemon, cinnamon, or ginger as the taste-masking agent, wherein the concentration thereof in the composition is from about 0.02 to about 2% w/w of the composition

38. A GI-cleansing composition according to claim 27, wherein the concentration of water is from about 15 to about 60% w/w of the total weight of the composition.

39. A GI-cleansing composition according to claim 27, wherein the concentration of water is from about 20 to about 60% w/w of the total weight of the composition.

40. A GI-cleansing composition according to claim 27, wherein the concentration of water is from about 25 to about 60% w/w of the total weight of the composition.

41. A GI-cleansing composition according to claim 27, wherein the gelling or swelling agent is selected from agar, guar gum, carrageenan, locust bean gum, acacia gum, xanthan gum, karaya gum, tara gum, and mixtures thereof.

42. A GI-cleansing composition according to claim 27, wherein the gelling or swelling agent is selected from gellan, pectin, konjak, and mixtures thereof.

43. A GI-cleansing composition according to claim 27, wherein the gelling or swelling agent is selected from gellan, agar, pectin, and mixtures thereof.

44. A GI-cleansing composition according to claim 27, wherein the gelling or swelling agent is selected from carrageenan, locust bean gum, and mixtures thereof.

45. A GI-cleansing composition according to claim 27, wherein the taste-improving agent is a laxative.

46. A GI-cleansing composition according to claim 27, wherein the taste-improving agent is selected from prune extract, plum extract, fig extract, coffee extract, and mixtures thereof.

47. A GI-cleansing composition according to claim 27, wherein the laxative is selected from PEGs having a molecular weight of from about 2000 to about 8000.

48. A GI-cleansing composition according to claim 27, wherein the laxative is PEG 3350.

49. A method of treating or alleviating constipation, comprising administering a composition according to claim 27 to a subject in need thereof.

50. A method of endoscopy, comprising administering a composition according to claim 27 to a subject in need thereof.

51. A method of treating an overweight subject, comprising administering a composition according to claim 27 to a subject in need thereof.

52. A method of treating obesity, comprising administering a composition according to claim 27 to a subject in need thereof.
